(19) United States
(12) Patent Application Publication
(10) Pub. No.: US 2008/0193329 A1
(43) Pub. Date: Aug. 14, 2008

(54) Method and System for Plasma Treatment Under High Pressure

(75) Inventors: Yuri S. Akishev, Moscow Region (RU); Anatoly P. Napartovich, Moscow Region (RU); Nikolay I. Trushkin, Moscow Region (RU); Christophe Leys, Evergem (BE)

Correspondence Address: CLARK & ELBING LLP, 101 FEDERAL STREET, BOSTON, MA, 02110, US

(73) Assignee: Universiteit Gent, Gent (BE)

(21) Appl. No.: 11/909,424

(22) PCT Filed: Mar. 21, 2006
(86) PCT No.: PCT/EP2006/002561

§ 371 Date: Sep. 21, 2007

(30) Foreign Application Priority Data
Mar. 21, 2005 (EP) 05447062.0

(51) Int. Cl. A61L 2/14; C23C 16/44; H05H 1/24

(52) U.S. Cl. 422/22; 422/186.04; 118/723 R; 427/569

(57) ABSTRACT

A method and system is provided for plasma treatment of material (202) at high pressure. The system used comprises a plasma generating means which comprises at least a first electrode (206) and a second electrode (208) for generating a plasma (204) pinned between the first electrode (206) and the second electrode (208) and a means (210) for displacing part of the plasma (204) towards a treatment area of said material (202). In this way the plasma (204) and the material (204) can interact, whereby the corresponding plasma current is substantially parallel with the treatment area of the material (202) to be treated. The means (210) for displacing may e.g. be a flow inlet system providing a flow of flow material (212) flowing substantially perpendicular to the direction of the plasma current in the part of the plasma (204) interacting with the material (202) to be treated. In this way efficient plasma treatment is obtained.

### TECHNICAL FIELD OF THE INVENTION

The present invention relates to a method and system for plasma treatment. In particular, the invention relates to a method and system for non-thermal plasma treatment of material at high pressure, e.g. 0.1 atmospheric to 2 times atmospheric pressure. The plasma treatment may e.g. be used in surface treatment of materials and in gas cleaning.

### BACKGROUND OF THE INVENTION

At present non-thermal plasma is a recognized instrument for surface treatment of different materials, such as e.g. films, fabrics, paper, etc. For instance, changing of wettability and adhesion, coating of surfaces, sterilization, and many other applications can be realized with the use of non-thermal plasma treatment.

In a non-thermal plasma (NTP) a processing gas, e.g. air, within NTP cells changes to a plasma state in the sense that electrons are liberated from the atoms and molecules of the processing gas. However, this liberation is not obtained because of the application of thousands of degrees Celsius; it is rather a high voltage, medium frequency, wave shaped electrical field that frees the electrons, the latter without forming a high temperature electric arc. Once the electrical field is removed, the electrons settle back into almost the same thermal state they were prior to the electrical excitation. When a pollutant or a surface to be treated is brought into contact with ionized processing gas, plasma-chemical reactions occur causing the pollutant or the surface to be reduced and/or oxidized or to obtain new properties.

In general, NTPTs can be divided in two groups: low-pressure NTPT, for which the pressure of the processing gas is lower than 0.1 atm and high-pressure NTPT, for which the pressure of the processing gas is larger than 0.1 atm, such as e.g. about 1 atm. Low pressure allows a more or less simple generation of diffusive (homogeneous) plasma with a volume of up to 10-30 litres. A further increase of the plasma volume requires the use of much more sophisticated and expensive gas discharge techniques. A schematic representation of a typical system 100 used for low pressure NTPT is shown in FIG. 1. In a vacuum chamber 102, preferably with a large size in order to be able to process a significant amount of material at the same time, a non-thermal plasma 104 is generated, using a plasma generator 106 for its excitation. The vacuum chamber 102 is brought under vacuum using a vacuum equipment 108. The material load 110 and material unload 112 typically is based on batch processing. The main drawback of low-pressure NTPT is the necessity to use (large-sized) vacuum chambers 102, which need a high quantity of metal for their fabrication, and the corresponding necessity for expensive vacuum equipment 108. Moreover, the batch processing does not allow continuous treatment of material, leading to the necessity to alternatively bring the vacuum chamber 102 at atmospheric pressure and under vacuum, which is time consuming. All this results in a poor compatibility of low-pressure NTPT with real industrial production lines.

These problems are limited or do not even occur when high pressure NTPT is performed. High-pressure NTPT, especially NTPT at atmospheric or higher pressure, allows for elimination of expensive vacuum equipment. A schematic representation of a typical system 150 that can be used for high-pressure NTPT is shown in FIG. 2. A plasma 104 is generated in a gas discharge chamber 152 at atmospheric pressure, connected with a plasma generator 106 for excitation of non-thermal plasma 104. The material to be treated is positioned outside the discharge chamber 152 and material load 110 and material unload 112 can be performed continuously, e.g. using a continuous film or a web 154. This system, allowing to perform continuous plasma processing, is more compatible with industrial production environments.

In operation, the main task of non-thermal plasma technology is the generation of chemically active species, such as e.g. radicals, ions or photons, that will react with a surface in a proper way. At atmospheric pressure, the lifetime of most active species is very short. It is therefore necessary either to provide a fast transfer of active species from the plasma region to the surface, or to produce active species immediately at the surface. One of both ideas is in one form or another used in all known approaches for the generation of non-thermal plasma at atmospheric pressure.

The first idea, i.e. wherein active species are transferred in a very fast way to the surface of the material to be treated, is used in so-called “remote surface treatment”, in which a cold or non-thermal plasma jet with active species is blown out of the plasma source onto the treated surface. Remote treatment is performed with plasma sources such as “plasma torches”, “plasma pencils” or a diffusive glow discharge at atmospheric pressure, as e.g. described in WO 02/09482. Remote surface treatment works well for many applications, but it does not provide an optimal use of the active species that are produced in the plasma source, due to inevitable losses during their transport by gas flow. Therefore, remote surface treatment might yield unsatisfactory results for those surface treatments that require high concentrations of active species.

A solution for the latter problem is obtained by using plasma sources based on the second principle, i.e. based on the generation of active species immediately at the surface. A first example thereof is industrially applied corona treatment apparatus wherein the plasma is indeed generated in the immediate vicinity of the surface. In modern corona treatment apparatus, typically there is no corona discharge but there is an alternating-current barrier discharge at typical frequencies of 10-40 kHz. A typical sketch of a corona treatment apparatus 175 is shown in FIG. 3. The corona treatment apparatus 175 shown has two barrier electrodes 180, covered with a dielectric, e.g. ceramic layer 182. A plasma current is generated between a barrier electrode 180 and a material support 190 functioning as second electrode, through the treated material 186. When viewed with the naked eye, the plasma 104 or gas discharge in the discharge gap 184 between the barrier electrodes 180 and the surface of the treated material 186 seems to be homogeneous. It is however well known that a barrier discharge in air under substantially atmospheric pressure consists of numerous thin (100 micron) current filaments 188, also known as streamers 188, which appear randomly over a short time during each half period and are chaotically distributed in the gap 184. These streamers 188 create non-thermal plasma 104 in the bulk of the discharge gap 184 and on the surface of the treated material 186 at the streamer contact points. The typical distance between neighbouring streamers 188 typically is roughly equal to the width of the discharge gap 184, which may e.g. be about 5 mm. The typical size of the current spot at the contact point of the streamer with the surface is markedly smaller than the gap length and moreover decreases with frequency f. This means that in corona treatment apparatus 175 the non-thermal plasma 104, and the corresponding active species, is mainly generated in the bulk of the discharge gap 184 and not at the surface of the material to be treated. Therefore, as a plasma source for surface treatment, the barrier discharge actually bears some similarity with remote plasma sources. Consequently the barrier discharge of corona treatment apparatus is not optimized for, or the most economical way of generating active species for surface treatment.

An alternative solution is described in US 2004/0194223, showing a dielectric barrier discharge multi-electrode system wherein the electrodes are embedded in a dielectric plate. The plasma thereby is generated using two coplanar electrodes consisting of parallel strips which alternate with respect to each other. The material is pressed on the surface of the dielectric material, where it is influenced by the active species of the plasma. The system has the disadvantage that a high voltage AC field is needed, which may result in the occurrence of sparks and furthermore that a dielectric material with a high dielectric permittivity needs to be used. The overall treatment obtained with this system may lack homogeneity, i.e. the surface treatment typically is in homogeneous.

In barrier discharge applications wherein the electrical current is oriented perpendicular to the surface and passes through the surface, damage can be caused to the material that is treated. For instance, in the case of a fabric consisting of dielectric fibres, most of the electrical current flows through the holes in the fabric and therefore current density in the holes can drastically increase. This concentration of the current density may lead to the destruction of the fabric.

In NTPT systems, control of different properties such as e.g. the type of discharge that occurs, is an important issue related to the efficiency of the system. In Appl. Phys. Lett. 83 (2003) 5392, Mase et al. describe a method for controlling the plasma production by capacity-coupled multi-discharge under atmospheric pressure. The system describes a system comprising a number of electrodes coupled directly with small capacitors for quenching the discharge. The use of capacity-coupled multi-discharge allows to obtain an efficient charge transfer for the single-pulsed discharge and a high efficiency of ozone production. However, capacity-coupled multi-discharge can work only in AC regime, not in DC, and can be used only as plasma source for remote surface treatment, with all drawbacks mentioned above and inherent in this approach.

### SUMMARY OF THE INVENTION

It is an object of the present invention to provide an improved system and method for high-pressure non-thermal plasma treatment of material. An advantage of the present invention is a high efficiency allowing to achieve the object at very low, reduced, power density, for instance, 0.1 W/cm² in the case of surface treatment. A further advantage is that a high degree of control can be obtained over the properties of the generated plasma.

The above objective is accomplished by methods and devices according to the present invention.

In a first aspect, the present invention relates to a device for high pressure, e.g. above 0.1, such as above 0.5 atmospheres, non-thermal plasma treatment of a material, the device comprising a plasma generating means comprising at least a first electrode and a second electrode for generating a plasma current and a plasma pinned between said first electrode and said second electrode and said device comprising a plasma displacement means for displacing part of said plasma current towards a longitudinally extending surface of an interaction area, e.g. treatment area of said material, where said material is treated, so that said plasma current and said material interact and that a direction of said plasma current interacting with said material is substantially parallel with said surface of said interaction area, e.g. said surface of said treatment area, where said material is treated. The interaction area may be a treatment area of said material to be treated.

The plasma may be a streamer-based plasma. Said plasma pinned between said first and said second electrode may have fixed points of application with respect to said first electrode and said second electrode during said displacing of part of said plasma current, or in other words during the application of a displacement by said displacement means. In other words for the plasma or part thereof, no change in points of application with respect to said first electrode and said second electrode may occur during said application of said displacement means. Said displacing may be performed during the complete high pressure, e.g. above 0.1, such as above 0.5 atmospheres, non-thermal plasma treatment of the material.

Said plasma generating means may comprise a DC high voltage generating source. Such a high voltage generating source may be adapted for generating a plasma in DC power regime.

At least one of said first electrode or said second electrode may be positioned freely. The wording “positioned freely” thereby means that the electrode is not embedded in solid material.

The plasma displacement means for displacing the plasma current towards an interaction area, e.g. treatment area of the material, where said material is treated may be an active means for displacing the plasma current towards an interaction area, e.g. treatment area, where the material is treated. The wording “active means” thereby refers to a means that by switching on allows to selectably or controllably displace at least part of the plasma.

The device furthermore may comprise a means for providing an area of said material to be treated in the vicinity of said electrodes.

The plasma displacement means may comprise a flow material inlet system allowing to provide a flow material flowing substantially perpendicular to the direction of said plasma current.

The plasma displacement means may comprise a means for generating an electric field. The plasma displacement means may comprise a means for generating a magnetic field.

The material to be treated may be a solid, continuous web-like material.

The material to be treated may have a relative rate of movement larger than 4 m/s, preferably larger than 7 m/s, more preferably larger than 9 m/s with respect to said plasma generating means.

At least one of the first electrode or said second electrode may be a resistive electrode.

The first electrode may comprise a plurality of electrode elements.

The first electrode and the second electrode may form a multi-pin to plate electrode system. The first electrode and the second electrode may form a multi-pin to multi-pin electrode system.

The plasma generating means furthermore may comprise at least one RC network coupled to at least one of said electrode elements. A resistor R in the RC network may be selectable in order to select between different types of plasma discharge. The resistor R may be a variable resistor.

The interaction area may be created by positioning a longitudinally extending surface of a plate in the vicinity of the electrodes, whereby said material to be treated is a gaseous flow directed towards said plate. The plate may comprise a catalyst.

In a second aspect, the invention also relates to a method for high pressure, e.g. above 0.1, such as above 0.5 atmospheres, non-thermal plasma treatment of a material, the method comprising creating a plasma discharge pinned between a first electrode and a second electrode of a plasma generating means, and displacing said plasma discharge so that it interacts in an interaction area, e.g. a treatment area of said material, with said material to be treated and that a direction of said plasma current interacting with said material is substantially parallel with a surface of said the interaction area, e.g. treatment area.

The plasma may be a streamer-based plasma. Said plasma pinned between said first and said second electrode may have fixed points of application with respect to said first electrode and said second electrode during said displacing of part of said plasma current, or in other words during the application of said displacement means. In other words for the plasma or part thereof, no change in points of application with respect to said first electrode and said second electrode may occur during said displacing. Said displacing may be performed during the complete high pressure, e.g. above 0.1 such as above 0.5 atmospheres, non-thermal plasma treatment of the material.

Creating a plasma discharge may be performed in a DC power regime, i.e. by means of a DC high voltage generating source.

The method furthermore may comprise providing said material to be treated near said electrodes. The step “providing said material to be treated near said electrodes” may comprise moving said material at a relative rate of movement with respect to said plasma generating means.

Displacing said plasma discharge may comprises generating any of an electric or a magnetic field near said plasma, said electric or magnetic field displacing part of said plasma discharge outside the region between the first electrode and the second electrode.

Displacing said plasma discharge may comprise providing a flow material flow onto said plasma substantially perpendicular to said plasma current.

Said material to be treated may be a gas. Said interaction area may be an area of a blocking material. Said flow of said gas may be directed to said blocking material. Said blocking material may comprise a catalyst in said interaction area. Displacing of the plasma discharge may be performed by said flow of said gas.

In a third aspect, the present invention also relates to a device for high-pressure plasma treatment of a material, the device comprising a plasma generating means, comprising at least a first electrode and a second electrode, at least one of them comprising a plurality of electrode elements, for generating a plasma between said electrode, wherein said plasma generating means comprises an RC network coupled to at least one electrode element of the first electrode or the second electrode.

The device furthermore may comprise a means for providing said material, thus allowing interaction between the material and the plasma. The means for providing said material is a means for providing a material flow guided through said plasma.

The means for providing said material may be a means for providing a solid material in the neighbourhood of said first and second electrodes.

The resistor R in said RC network may be selectable in order to select between different types of plasma. The resistor R may be a variable resistor R.

In a fourth aspect, the invention furthermore relates to a method for high-pressure plasma treatment of a material, the method comprising selecting at least one resistor value in at least one RC network coupled to at least one electrode element of a first electrode or a second electrode, according to a type of plasma to be obtained, generating a plasma between at least a first electrode and a second electrode, at least one of said electrodes being connected to said RC network, providing said material thus allowing interaction between said material and said plasma. Selecting at least one resistor value in at least one RC network may comprise selecting and setting a value of a variable resistor.

It is an advantage of the present invention that a system and method for high-pressure non-thermal plasma treatment of material according to embodiments of the present invention allow for highly homogeneous treatments of materials.

It is also an advantage of the present invention that a system and method for high-pressure non-thermal plasma treatment of material according to embodiments of the present invention have a good thermal stability. As in different embodiments of the system inherently a cooling mechanism is applied and due to the high efficiency of the system, the thermal regime in which the system can be operated and the method can be applied is significantly better than in prior art systems, where no cooling methods are present and wherein furthermore additional cooling methods cannot be applied easily or efficiently.

It is an advantage of the embodiments of the present invention that an intensive and chemically active non-thermal plasma can be created immediately at the surface of material to be treated, and optimally can be used for treatment of material.

It is also an advantage of the present invention that there is no limit on the thickness of the NTPT treated material and that the thickness of the material to be NTPT treated can be significantly larger than the thickness of material when prior art systems or techniques are used.

It is an advantage of the embodiments of the present invention that intensive and chemically active plasma can be created by use of different kind of power supplies, such as e.g. an AC high-voltage generator, a DC high voltage generator or a pulsed periodical high-voltage generator.

It is furthermore an advantage of the present invention that the system for high-pressure non-thermal plasma treatment has an increased lifetime.

It is also an advantage of the present invention that the properties of the discharge created are significantly independent or decoupled from the materials used for the setup and the materials to be NTPT treated.

It is also an advantage of the present invention that it can be exploited, if necessary or wanted, for remote plasma treatment and for treatment by surface streamers creating non-thermal plasma immediately at the treated surface. Transition from one option to another may be made by varying the distance between the outlet edge of a discharge and the material to be treated.

It is furthermore an advantage of embodiments of the present invention that the rate at which material can be treated can be significantly higher than in other high-pressure NTPT treatments. The direction of the material transport can be realized both along as well as transverse to the electric current in the surface streamers.

It is an advantage of the embodiments of the present invention, that the breakdown voltage for the generation of surface streamers is lower than that for the initiation of volume streamers in dielectric barrier discharges, thus resulting in a reduced environmentally harmful ozone production during the discharge.

Particular and preferred aspects of the invention are set out in the accompanying independent and dependent claims. Features from the dependent claims may be combined with features of the independent claims and with features of other dependent claims as appropriate and not merely as explicitly set out in the claims.

Although there has been improvement, change and evolution of devices in this field, the present concepts are believed to represent substantial new and novel improvements, including departures from prior practices, resulting in the provision of more efficient, stable and reliable devices and methods of this nature.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a schematic representation of a system that can be used in low-pressure non-thermal plasma treatment, as known from prior art.

FIG. 2 is a schematic representation of a system that can be used in high-pressure non-thermal plasma treatment, as known from prior art.

FIG. 3 is a schematic representation of a system for providing corona treatment, as known from prior art.

FIG. 4 is a schematic illustration of a side view of a system for providing an efficient high-pressure non-thermal plasma treatment of a surface material, according to a first embodiment of the present invention.

FIG. 5 is a schematic illustration of a top view of the system for providing an efficient high-pressure non-thermal plasma treatment of a surface material shown in FIG. 4, the system being illustrated without the displacement means.

FIG. 6 is a side view of a system comprising a flow material inlet system according to a specific example of a first embodiment of the present invention.

FIG. 7 is an enlarged side view of a system for providing an efficient high-pressure non-thermal plasma treatment of a material as shown in FIG. 4, the system being shown without the displacement means.

FIG. 8 is a specific example of a multi-pin to plate system for plasma treatment of a material, according to a second embodiment of the present invention.

FIG. 9 is a detailed view of part of a multi-pin to plate system for plasma treatment of a material, as shown in FIG. 8.

FIG. 10 is a schematic representation of a system comprising additional electronics for performing efficient plasma treatment of solid and gaseous materials, according to a third embodiment of the present invention.

FIG. 11 is a schematic representation of an system comprising additional electronics for performing efficient plasma treatment of a material flow, according to a third embodiment of the present invention.

FIG. 12a and FIG. 12b are a schematic representation of a side view respectively a photographic image of a top view of a system for providing an efficient high-pressure non-thermal plasma treatment of a surface material, according to an example of the present invention.

FIG. 13 and FIG. 14 illustrate the hydrophilicity of a non-treated respectively treated polypropylene strip for a non-thermal plasma treatment as applied according to an example of the present invention.

FIG. 15 shows the contact angle for a drop of liquid on a non-thermal plasma treated narrow polypropylene strip (width 1.5 mm) as a function of the treatment time during a non-thermal plasma treatment applied according to an example of the present invention. The plasma processing gas is ambient air at atmospheric pressure.

FIG. 16 shows the contact angle for a drop of liquid on a non-thermal plasma treated broad polypropylene strip (width 5.5 mm) as a function of the velocity of the polypropylene strip for a non-thermal plasma treatment applied according to an example of the present invention. FIG. 16 also indicates that the ageing effect, i.e. the increase in contact angle of the treated strip with time, after treatment is small.

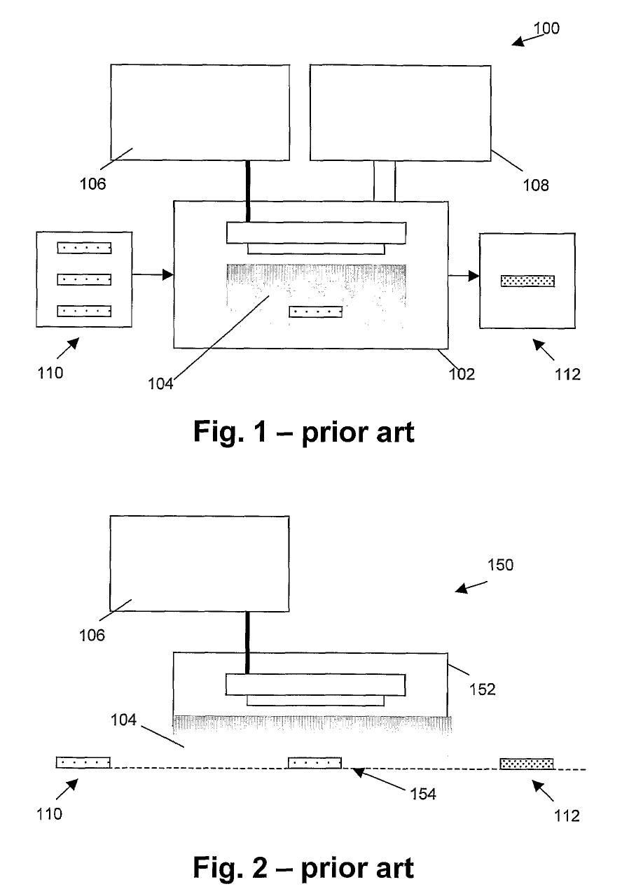

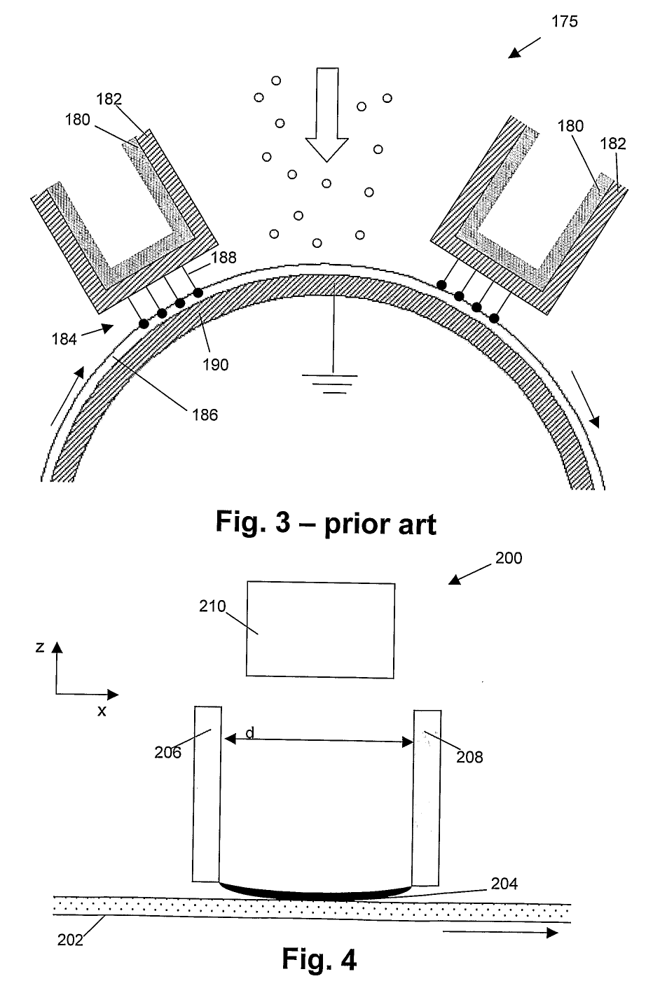

In the different figures, the same reference signs refer to the same or analogous elements.

### DESCRIPTION OF ILLUSTRATIVE EMBODIMENTS

The present invention will be described with respect to particular embodiments and with reference to certain drawings but the invention is not limited thereto but only by the claims. It is clear that other embodiments of the invention can be configured according to the knowledge of persons skilled in the art without departing from the true spirit or technical teaching of the invention. The drawings described are only schematic and are non-limiting. In the drawings, the size of some of the elements may be exaggerated and not drawn on scale for illustrative purposes. The dimensions and the relative dimensions do not correspond to actual reductions to practice of the invention.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Similarly, it is to be noticed that the term “coupled”, also used in the claims, should not be interpreted as being restricted to direct connections only. Thus, the scope of the expression “a device A coupled to a device B” should not be limited to devices or systems wherein an output of device A is directly connected to an input of device B. It means that there exists a path between an output of A and an input of B which may be a path including other devices or means.

With the term “high pressure” typically a pressure equal to or higher than 0.1 atmosphere is meant. The systems and methods for high-pressure NTPT treatments thus operate at a pressure equal to or higher than 0.1 atmosphere, preferably higher than 0.5 atmosphere, more preferably about atmospheric pressure or higher than atmospheric pressure. Preferably NTPT treatments are operated at a pressure smaller than 2 atmospheres, more preferably at substantially atmospheric pressure, i.e. under atmospheric pressure.

The term “plasma” is used to identify gaseous complexes which may comprise electrons, positive or negative ions, gaseous atoms and molecules in the ground state, radicals or any higher state of excitation including light quanta. The most common method for achieving a plasma state is through an electrical discharge.

With “non-thermal” plasma, also called “cold” plasma or “non-equilibrium” plasma, a plasma is meant wherein the electron mean energy is much higher than the ion and gas mean energies. The plasmas thus comprise gas atoms at room temperature and electrons at much higher temperatures, i.e. typically several tens of thousands Kelvin. This plasma state provides an ambient gas temperature along with electrons which have sufficient kinetic energy to cause the cleavage of chemical bonds. As a result, cold plasmas are highly suitable for chemical reactions, such as gas clean up and odour control, organic synthesis, polymerizations and various treatments, such as surface treatments including sterilization of bio-medical materials and instruments. Cold plasmas are characterized by average electron energies of 1 eV to 10 eV and electron densities of 10⁹ to 10¹⁵ cm⁻³. Other synonymous terms for cold plasma are “glow discharge” or “low temperature plasma”. In the glow discharge, electrons are produced in the gas phase by ionization of neutral species by electrons accelerated by the electric field. Generally, the electrodes are not consumed in a glow discharge, although the invention is not limited thereto.

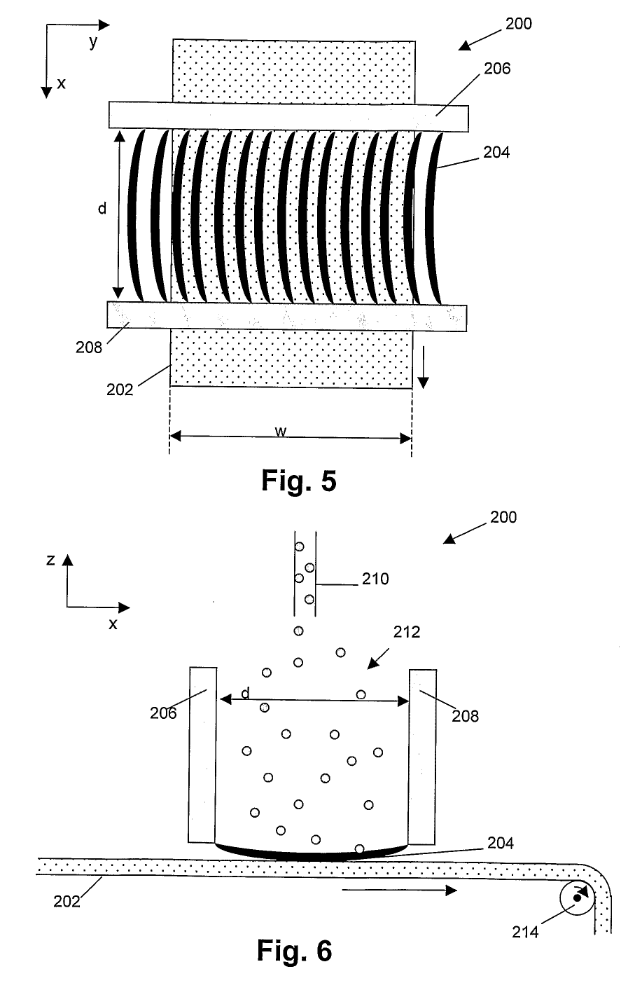

In a first embodiment, the present invention relates to a method and system for generation of a non-thermal plasma for treatment of material. The different components of the system are shown in FIG. 4 and FIG. 5, illustrating a side view respectively top view of a schematic representation of a system 200 for non-thermal plasma technology (NTPT) treatment of a material 202, i.e. plasma treatment of a material 202 using a non-thermal plasma 204. The material to be treated may for example be, but is not limited to, metals, ceramics, glass, polymers, circuit boards, biomedical devices, instruments and materials, textiles. The surface treatment to be carried out may for example be, but is not limited to,
surface cleaning—such as e.g. removal of organic surface contamination from materials that require critical cleaning,
adhesion promotion—such as e.g. for improvement of bondability of a second surface or material to the material to be treated e.g. by creating chemically active functional groups on the surface such as amine, carbonyl, hydroxyl and carboxyl groups,
control of surface energies—such as e.g. creating hydrophilic or hydrophobic surfaces,
improvement of biocompatibility—such as e.g. for biomaterials that come into contact with blood or protein,
surface sterilization—such as e.g. prompt removal of the dangerous consequences of bio-terrorism attack (killing the hard-died bacteria and microbes on the wall, etc),
gas-phase deposition of the needed thin layers on the surface or coating the surfaces using flow of the liquid aerosol precursors delivering high chemical functionalities onto the substrate.
enhancing performance of materials in certain applications—such as e.g. by increasing hardness and/or chemical resistance of the materials.

The system of the first embodiment of the present invention comprises plasma-generating means (not shown completely) comprising at least a first electrode 206 and a second electrode 208 and a plasma displacement means 210 for providing a displacement of generated plasma towards the material 202 to be treated. The plasma generating means may use a DC or an AC power supply for the excitation of the plasma 204. Consequently, the plasma generating means may furthermore comprise typical DC, AC or pulsed periodical driving electronics for generating a field between the first electrode 206 and the second electrode 208, as well known by a person skilled in the art. A processing gas present between the first and second electrodes 206, 208 may be used for generating the plasma 204. The plasma may be a streamer-based plasma. The first electrode 206 and the second electrode 208 each may be extending substantially in a single plane, such as e.g. in a setup of two parallel plates or in a setup wherein a number of pins, needles or edges are equally spaced from a plate electrode, although the invention is not limited thereto. The first electrode 206 and the second electrode 208 thus may be positioned opposite to each other, i.e. not coplanar, spaced by a gap, and preferably in planes substantially parallel to the plane of the material to be treated. In an alternative embodiment, not represented in the drawings, the first and second electrodes can be co-planar electrodes, i.e. electrodes lying in a single plane substantially parallel to the plane of the surface to be treated, the electrodes having a gap in between them. If the electrodes are sectioned electrodes comprising a plurality of electrode elements the electrode elements of the co-planar electrodes may be interdigitated, i.e. anodes and cathodes alternate each other. In this case, the total discharge will be discrete and it will consist of a number of short current filaments oriented along a single straight line belonging to the plane of co-planar electrodes. Compared to these systems, systems with opposite positioned electrodes allow to provide a thicker layer of plasma which e.g. advantageously allows to treat more rough materials such as e.g. textile.

The generated plasma typically is pinned at the first electrode 206 and the second electrode 208. In other words, the points of application of the plasma with respect to the first electrode 206 and the second electrode 208 may be fixed and furthermore may stay fixed during the application of the displacement means. The points of application of the plasma with respect to the first electrode 206 and the second electrode 208 thus may be fixed during the complete treatment process of the material. At least one of the first electrode 206 and the second electrode 208, and more preferably both the first electrode 206 and the second electrode 208 may be positioned freely, i.e. not embedded by another solid material. The latter may allow an improved thermal stability of the system. The first electrode 206 and the second electrode 208 may be standard electrodes used for plasma generation such as e.g. metallic electrodes. Alternatively, one of the first or second electrodes 206, 208, preferably the non-active electrode, also may be a resistive electrode. The plasma displacement means 210 may be any means that allows to actively displace at least part of the plasma. Part of the plasma is displaced from between the electrodes 206, 208 such that interaction with a material provided near the electrodes 206, 208 but not in between the electrodes 206, 208 is obtained. With “actively displacing” is meant that the displacement occurs when the plasma displacement means 210 are switched on and not when the plasma displacement means 210 are switched off. These plasma displacement means 210 will be discussed in more detail below.

The system 200 may also comprise a material providing means for providing the material 202 to be treated or influenced by the generated non-thermal plasma 204. This material providing means typically is a system for providing material 202 near the plasma 204, such as e.g. a system for providing a continuous material or web-like material like film, thread, etc. which may e.g. be a system based on rollers 214, a system such as a conveyor belt whereon material can be provided or a support system whereon material, such as e.g. non continuous material can be placed. It is an advantage of the present system that a continuous operation of the system can be provided whereby a high quality material treatment, such as e.g. a surface treatment, can be provided. The material providing means may comprise a means for supporting the material 202 to be treated, thus preventing that the material 202 is displaced by the action of the plasma displacement means 210. As the material providing means does not function as a second electrode, and hence does not need to collect the electrical current of the plasma, the material providing means may be made from a cheap and light material such as e.g. plastic.

The material 202 to be treated is positioned near the electrodes 206, 208 of the system 200 such that the plasma 204 can interact with at least part of the material 202, that part being referred to as a treatment area. The treatment area may be e.g. the surface of the material 202, but it may also be the top region of the material 202, i.e. a kind of skin, deeper than the surface, or, depending on the thickness of the material 202 provided, the whole material. Depending on the permeability of the material 202 to be treated for the active species of the plasma 204, material treatment over a depth of 0.01 mm, 0.1 mm, 1 mm, 10 mm up to 35 mm can be obtained. This thickness is determined by geometrical properties of the material to be treated, by the displacement force applied, e.g. determined by the gas-flow velocity, and by the life-time of the active species created by the plasma in the vicinity or inside of such material. It is to be noted that, in case of non-porous materials, the plasma only treats a thin, upper layer of the material, whereas in case of porous material the thickness of the treatment layer is determined by the depth of penetration of the active plasma into the material. The latter can be obtained as the active species permeate into the material 202 based on convection rather than on diffusion. The non-porous material thus in principle does not have a limited thickness, as only the upper layer is treated, while porous material should be limited in thickness.

The degree of treatment of the material 202 is influenced by the duration of the treatment, which for moving material 202 depends on the velocity or rate of movement of the material 202 along the plasma 204 and the properties of the plasma 204. The efficiency of treatment with the plasma 204 is determined by the frequency for the generation of aerodynamically retained surface streamers. This frequency can be increased up to 300, preferably up to 400 kHz. Using such a frequency in treatment lines for treating material allows to reach a line speed of up to 30 m/s for the material 202 to be treated.

Typically, the material 202 to be treated is not positioned in between the electrodes 206, 208, but in a region in the vicinity of the electrodes 206, 208. Preferably the material 202 to be treated is positioned such that no additional solid material, such as e.g. electrode material or other solid material, is present between the treatment area of the material 202 to be treated and the gap between the electrodes 206, 208. The material 202 to be treated may therefore be positioned substantially perpendicular to the planes determined by each of the electrodes 206, 208. The material may preferably be positioned/moved in the vicinity of the electrodes 206, 208, along or in an x,y plane indicated in FIG. 5, for electrodes 206, 208 substantially extending in the y-direction or in the y- and z-direction as indicated in FIG. 4. If, for continuous material 202 such as e.g. web material, the material 202 is moved along the x-direction indicated in the co-ordinate systems of FIG. 4 and FIG. 5, simultaneous treatment of materials 202 having a large width w is allowed. The latter is indicated in FIG. 5. The width w of the system 200 is determined by the length of the electrodes 206, 208 in the y-direction and is in principle not limited. Consequently, neither is the width w of the material 202 that can be simultaneously treated limited. It is to be noted that in the drawings, and in particular in FIG. 5, the distance d between the electrodes 206, 208 in the x-direction is strongly exaggerated compared to the width w of the material 202 that can be simultaneously treated. If the material 202 is moved along the y-direction of the co-ordinate system indicated in FIG. 5, the width w of the material that can be treated simultaneously using a single system as shown in FIG. 4 and FIG. 5, is limited by the width d of the gap between the two electrodes 206, 208. Positioning or moving the material 202 along the x,y-plane, guarantees that a high density of active species can be obtained at the surface of a material 202, as the plasma current of the part of the plasma 204 interacting with the material 202 is substantially oriented along, i.e. parallel with the surface of the material 202 to be treated. This is in contrast with prior art systems wherein the plasma current typically is passed perpendicular to the surface of the material to be treated. The generated power density that can be obtained with embodiments of the present invention may be up to 10 W/cm², preferably up to 40 W/cm², more preferably up to 75 W/cm², even more preferably up to 100 W/cm². It is to be noted that the material may be moved in any direction substantially parallel to the x-y plane—the x-y plane shown in FIGS. 5, 8, 9 and 10—without departing from the scope of the present invention.

Typical examples of plasma displacement means 210 will now be discussed in more detail. Such examples may be a flow material inlet system, an electric field generating means, a magnetic field generating means, etc.

A first example of a plasma displacement means 210 is shown in FIG. 6, showing a flow material inlet system as a plasma displacement means 210. In order to allow good interaction between the plasma 204 and the material 202 to be treated, at least part of the plasma 204 is displaced towards the material 202 to be treated by means of a generated flow material 212 flow. The plasma thereby still are pinned with some edges to the electrodes 206, 208. The flow rate necessary to substantially force the part of the plasma 204 towards the material 202 may be in a range having a lower limit of 1 m/s, preferably 5 m/s, more preferably 10 m/s and having a higher limit up to sonic speed. The flow material 212 flow is preferably oriented perpendicular to the plasma current, in order to allow the most optimum displacement capacity, although the invention is not limited thereto and any flow direction differing significantly from the plasma current direction between the electrodes 206, 208, i.e. differing significantly from the x-direction as shown in FIG. 6, allows displacement of part of the plasma 204. The flow material 212 flow furthermore ensures a homogeneous distribution of the active species of the plasma 204 over the material 202, e.g. the surface of the material 202, to be treated. Introduction of the flow material 212 flow can be done using the inlet system of plasma displacement means 210. This may e.g. be a pipe through which flow material 212 is provided. Typical flow materials 212 that can be used may be gasses such as ambient air, inert gasses such as e.g. nitrogen and argon, or any other gas that allows to force part of the plasma 204 towards the material 202 to be treated, without substantially influencing the plasma 204. Alternatively, gasses comprising materials that assist in the creation of a plasma also may be used. Other additives, which have a function in the treatment process may also be comprised in the flow material 212. A typical example thereof are water vapour and/or chemical aerosols, added in the flow material to perform surface coating of the material 202 due to liquid deposition assisted with plasma. It is an advantage for such applications that the additives contact the surface simultaneously with the plasma 204, which may result in a high efficiency of the treatment, e.g. coating process. The use of a flow of flow material 212 furthermore may allow that operation under a good thermal regime is obtained. In other words, the use of a flow material 212 flow, such as e.g. a gas flow, increases the thermal stability of the system.

A second example of a plasma displacement means 210 is an electric or magnetic field generating means providing an electric field or a magnetic field exerting a repulsive or an attractive force on the active species in the plasma 204, such that part of the plasma 204 is displaced from between the electrodes 206, 208. The plasma 204 thereby still may be pinned at the edges of the electrodes 206, 208. Use of any of the plasma displacement means 210 as described above, allows that almost 100% of the active plasma species are generated immediately near the material 202 to be treated, e.g. at the surface of the material 202 to be treated. The direction of the plasma current for the plasma 204 interacting with the material 202 thereby is substantially parallel with the material 202. The latter significantly increases the efficiency of the treatment process.

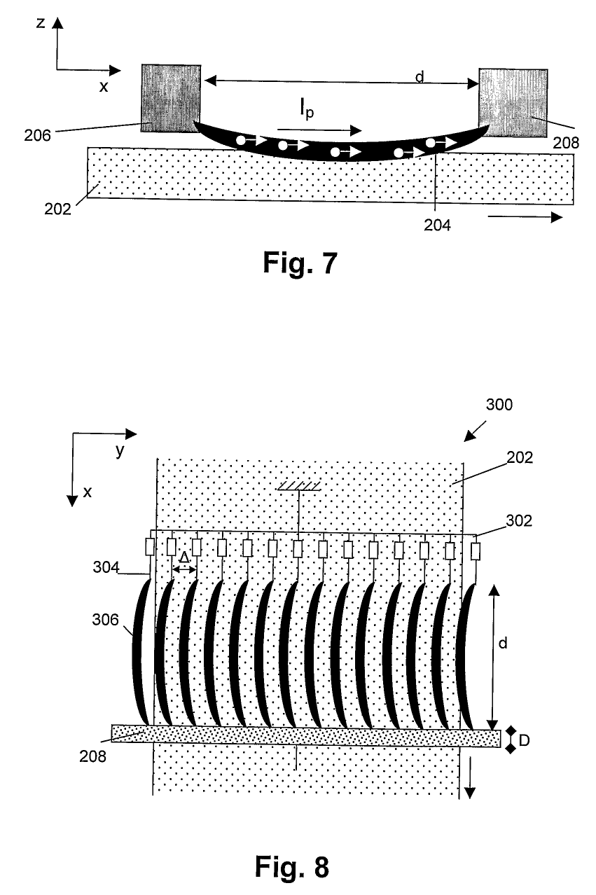

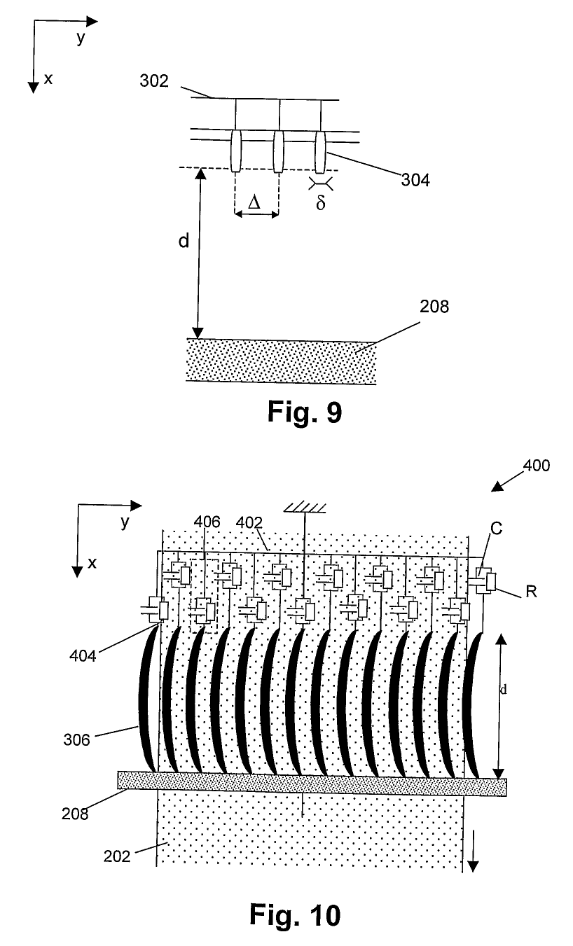

In the present embodiment, the non-thermal plasma thus is generated between the first electrode 206 and the second electrode 208 but is directed by external actuation means, i.e. an actuation not inherent to the plasma generation, towards the surface of the material 202 to be treated. The material 202 to be treated is provided adjacent the at least two electrodes 206, 208 such that the generated plasma current Iₚ flows substantially parallel to the surface of the material 202 to be treated, and not perpendicular to the surface of the material to be treated as is the case in prior art systems. The latter is shown in more detail in FIG. 7, showing a detailed image of the position of the material 202 to be treated with respect to the first and second electrodes 206, 208 and the direction of a plasma current flow in the plasma 204. The direction indicated by the arrow labelled Iₚ is the direction of movement of a plasma current Iₚ of charged particles. Depending on which of the electrodes 206, 208 is the cathode and which of the electrodes 206, 208 is the anode, the sense of the arrow indicating the plasma current Iₚ shown may represent the movement of the positively charged particles or the direction of the negatively charged particles. The latter only influences the sense of the plasma current Iₚ, not the direction of the plasma current Iₚ. If the material 202 to be treated is provided continuously, the direction of movement of the material 202 may be the same as or opposite to the direction of the plasma current Iₚ. Alternatively, the direction of movement of the material may be in any direction in the x,y-plane indicated in FIG. 5, FIG. 8, FIG. 9 and FIG. 10, e.g. perpendicular to the direction of the plasma current Iₚ.

In a second embodiment, a system having the same features as the first embodiment is described, whereby at least the first electrode is sectioned. In such a system, the generated plasma comprises numerous streamers along the whole length of the electrodes. At least the first electrode is sectioned into a number of small, sharp metallic elements. An exemplary system 300 is shown in FIG. 8 and part thereof in an enlarged view in FIG. 9. The system 300, shown by way of example, comprises a multi-pin electrode 302 and a second electrode 208, being a plate electrode. The multi-pin electrode 302 is sectioned into a number of small, sharp conductive, e.g. metallic, elements 304. Such small, sharpen metallic elements 304 may be e.g. needles, pins, knives, etc. The sharp metallic elements 304 thereby are electrically insulated from each other. Each of the sharp metallic elements 304 is able to generate a streamer 306 between the sharp metallic element 304 and the second electrode 208. By proper spacing of the sharp metallic elements 304, the streamers 306 are generated spaced at regular distances from each other. Individual ballast resistors (not shown in FIG. 8 and FIG. 9) may be provided for each of the sharp metallic elements 304. The second electrode 208 may be positioned opposite to the multi-pin electrode 302. Both a multi-pin cathode to plate anode set-up and a multi-pin anode to plate cathode set-up can be used. As the system is built up modularly, i.e. the sharp metallic elements 304 can be provided in a modular way, the system can easily be extended and thus easily allows up-scaling. The distance d between the free extremities of the sharp metallic elements 304 of the multi-pin electrode 302 and the plate electrode 208 typically is within the range having a lower limit of 0.1 mm, preferably 0.3 mm, more preferably 1.0 mm and having an upper limit of 5 cm, preferably 3.5 cm, more preferably 1.5 cm. The optimum distance d to be used depends on the needed level of plasma and current densities created at the surface which in turn depends on gas flow velocity, gas flow composition and properties of the material to be treated. Electrical and thermo-physical properties of the material to be used to fabricate the electrodes influence also the optimum distance d. Geometrical sizes of the sharp metallic electrodes influence the optimum distance d as well. The typical distance in the y-direction between neighbouring sharp metallic elements 304 of the sectioned, first electrode 302, i.e. typical distance Δ, may be in a range having an upper limit being 3*d, preferably 2*d, even more preferably 1.5*d and a lower limit 0.1*d, preferably 0.3*d more preferably 0.5*d, d being the width of the gap between the first and the second electrode. A typical transverse size in the y-direction of a single sharp metallic element 304 of the sectioned electrode equals δ, being in the range having an upper limit Δ, preferably 0.7*Δ, more preferably 0.5*Δ and a lower limit 0.01*Δ, preferably 0.05*Δ, more preferably 0.1*Δ. The curvature radius of the tip of a single sharpened metallic element can be ranged from several micrometers up to several millimetres. The distances d, Δ and δ are indicated in FIG. 9.

The second electrode 208 may be a plate electrode, but alternatively also may be a sectioned electrode, similar to the first sectioned electrode 302. Both plate electrodes and sectioned electrodes may be either made of resistive material or of conductive, e.g. metallic, material. In case of conductive sectioned electrodes, the sections are electrically insulated from each other. The specific shape of the second electrode 208 may e.g. be a planar shape, a substantially cylindrical shape, a cylindrical-like shape, such as e.g. a tube, etc. The typical width D or, in case of cylindrical or rounded electrodes, the diameter D of the second electrode 208 in the z-direction lies in a range having a lower limit 0.1 d and an upper limit 5 d. The length of the sectioned first electrode 302 and the second electrode 208 in the y-direction can be as long as is needed for the application. The latter thus may be adjusted to the width of the material 202 to be treated. Typically, one of the electrodes is grounded. The other electrode is fed by either steady-state or pulsed, e.g. periodically pulsed, direct current (DC) at high voltage or alternating current (AC) at high voltage. The more suitable regimes, which are also of greater practical interest due to the availability of low-cost and robust power sources, are the steady-state DC and AC regimes.

In the present embodiment, the non-thermal plasma is at least partly built up from a large number of streamers 306. The numerous streamers 306 are generated at high frequency, and are closely spaced in the gap between the sectioned, first electrode 302 and the second electrode 208. The streamers 306 are forced tightly against the material 202 to be treated by the plasma displacement means 210 (not shown in FIG. 8 and FIG. 9). In this case, almost 100% of the active plasma species, present in the streamers 306 in the plasma, are generated immediately at the material 202 to be treated. The plasma displacement means 210 also ensures a more homogeneous distribution of the active species over the material 202. It is to be noted that the treatment systems described in the present invention are based on modular discharge devices and therefore are scalable to industrial roll widths, whereby the uniformity of the treatment is maintained.

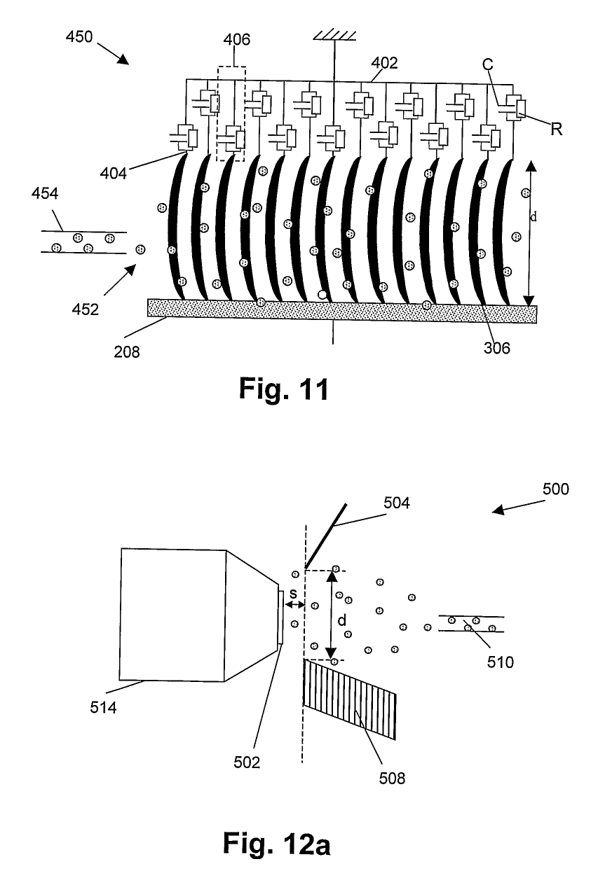

In a third embodiment, the invention relates to systems 400, 450 for treating material 202, 452, e.g. solid material 202 or gas 452, as shown in FIG. 10 and FIG. 11. The system 400, 450 comprises a plasma generating means comprising at least a first electrode 402 and a second electrode 208, whereby at least the first electrode 402 is sectioned into a number of small, sharp conductive, e.g. metallic, elements 404. Such small, sharp metallic elements 404 may be e.g. needles, pins, knives, etc. The sharp metallic elements 404 thereby are electrically insulated from each other. The second electrode 208 may be a conventional plate-like electrode, but also may be a sectioned electrode, similar to the first electrode 402. The sectioned first electrode 402 as well as the second electrode 208 may be the cathode of the system, while the other electrode then is the anode of the system. The plasma generating means also typically may comprise a driving means (not shown in FIG. 10 or FIG. 11) for generating plasma in DC, AC or pulsed-periodical regimes of power supply. The plasma generating means furthermore comprises a regulating system provided such that all streamers 306 generated in the multi-pin to plate system 400 or in the multi-pin to multi-pin system can be created simultaneously and repetitively, in the case of steady-state DC regime and AC or pulsed-periodical regimes of power supply. A regulating system comprises at least one RC-network 406 coupled in series with at least a number of sharp metallic elements 404 of the sectioned first electrode 402, preferably with each of the sharp metallic elements 404 of the sectioned first electrode 402 an RC-network 406 is coupled. The resistance of each resistor R in the RC-networks 406 preferably is relatively high and limits the current through the RC-network 406 to a level below the high current level that is needed to support the existence of a streamer 306 linked to the corresponding electrode element 404 to which the RC-network 406 is coupled. The capacitor C in each RC network is connected in parallel to the resistor R. Typical resistor values that can be used are in the range between 0.3 Mohm and 30 Mohm, although the invention is not limited thereto. As a result the streamer 306 exists only during the short time of gap breakdown. The time for which gap breakdown, and thus streamer existence, occurs is determined by the charging time of the capacitor C. Typical capacitor values that can be used for a standard set-up are in the range between 3 pF and 300 pF. Typical parameters obtained in such as system correspond to approximately 1 A for the current amplitude of a single streamer and a duration of about 0.1 to 0.5 μs. After breakdown, the capacitor C discharges through the resistor R, and the breakdown process associated with streamer generation repeats again. By choosing R and C in a proper way, it is possible to reach a high repetition frequency in the appearance of streamers 306 up to 300 kHz. This “DC” approach enables to obtain high frequency in generation of streamers 306 per electrode element without use of high frequency pulsed-periodical DC high voltage or AC high voltage excitation. The intensity of the generated plasma also can be controlled by controlling the electric parameters such as the resistor value R and the capacitor value C, thus changing the repetition frequency of the streamers 306 and the amplitude of their current. In other words, the type of discharge can be tuned by fitting an appropriate RC value. This allows to create different types of discharges. Another advantage is that the relatively low dose of energy released in the streamer 306 due to charging the capacitor C results in a restriction of the gas temperature inside the streamer 306. The system therefore allows the generation of mild streamers at a high frequency which are able to treat heat-sensitive samples with high efficiency.

In FIG. 10, a system 400 according to the present embodiment is shown that can be used to treat non-gaseous materials 202, e.g. solid materials, that are provided near the electrodes 402, 208 and by using a plasma displacement means (not shown in FIG. 10), for displacing the plasma consisting of the numerous streamers 306 from between the electrodes 402, 208 towards the solid material 202. The plasma displacement means may be a flow material inlet system creating a flow of flow material onto the plasma, a magnetic field generating means or an electric field generating means. The concept of a plasma displacement means and its function is in more detail described in the first embodiment.

The above-described embodiments of the present invention also can be used successfully to treat gaseous materials, i.e. cleaning gaseous material or controlling odour of gaseous material. This can e.g. be obtained by providing a blocking material, e.g. a static dielectric plate, instead of moving material and by providing a gaseous material flow towards the blocking material, e.g. dielectric plate. In this way, an intensive plasma is created at an interaction area of said blocking material, e.g. the static dielectric surface. In such a case, chemically active species generated by surface streamers react with harmful contaminants in gaseous material flowing over the blocking material. The interaction between active species and gaseous contaminants, such as e.g. harmful or smelly gaseous additives, at the surface allows to destroy the gaseous contaminants. Another and more interesting option for plasma treatment of gaseous materials, e.g. cleaning of gas or odour control, is based on a system using a blocking material as described above, whereby a proper catalyst is used instead of a blocking material, e.g. a normal dielectric plate. In this case, a synergetic effect between the plasma and the catalyst, which results in a strong increase in efficiency of the destroying smelly contaminants or harmful additives in the treated gas flow. For instance the plasma may be used in photocatalytic oxidation. Typical examples of such catalyst may be e.g. titanium oxide that can be used as the photocatalyst. This catalyst is activated by the plasma, e.g. by UV emissions from the plasma.

In another aspect of the present invention, a plasma generating system with an RC regulator as described above, but without a need for a plasma displacement means, can be used to treat gaseous materials, e.g. in a set-up 450 as shown in FIG. 11. The gaseous material 452 to be treated then is sent through the plasma between the first and second electrodes 402, 208 and interacts with the active species of the streamers 306 in the plasma. The gaseous material may e.g. be provided through a well-oriented inlet pipe 454, or be provided through an inlet and a fan, guiding the gas in between the electrodes 402, 208. The gas may be sent through the plasma in any direction, for example the y-direction or in the z-direction, or in a multi-pin to multi-pin set-up even in the x direction.

In the present embodiment, an optimization of the discharging thus is obtained by selecting an appropriate resistor R, in addition to the resistive value of the discharge unit as such, and a capacitor C. This optimization allows to obtain a high repetition frequency and an improved heat control of the system. The selection of an appropriate resistor R may be done e.g. during fabrication of the system. In a preferred embodiment variable resistors R may be provided, allowing to select an appropriate resistor for each plasma treatment, or even to change the resistance during the plasma treatment. The latter could e.g. also be used to perform treatment according to specific patterns on the material. By changing e.g. the resistance value for the different electrode sections, a plasma may be generated in specific patterns with respect to the material to be treated, and thus a plasma treatment may be performed in specific patterns. Such a system can operate e.g. similar to an image-wise printing system. The variable resistors may e.g. be set by a controller, controlling the settings for creating a plasma in accordance with the image by which the treatment needs to be performed.

In a preferred embodiment for any of the above-described systems, one of the electrodes may be a resistive electrode. The latter is especially useful to prevent the system from going into sparking regime. Transition from the streamer regime to the sparking regime typically occurs when the electrical power density in the discharge is increased beyond a critical level. Sparks are hot plasma channels with high current density that short-circuit the electrode gap. In contrast to streamers, sparks are ineffective for surface treatment or radical production. To increase the streamer-to-spark threshold power density, the second electrode 208 is preferably made from a resistive material. Alternatively, the second electrode 208 also can be coated with a resistive material. The resistive material typically has a high dielectric strength and a sufficient homogeneous volume resistivity. A large number of resistive materials can be used such as inorganic composite materials having a large dielectric strength, e.g. composite materials based on aluminum oxide or nitride-passivated ceramics, different types of glass, such as e.g. Soda-Lime glass, Pyrex and Vycor, and organic materials having a large dielectric strength such as materials comprising an adapted epoxy resin. From electrical point of view, the minimum thickness of the resistive material to be used, e.g. if a thin film is applied, depends on the spot size of the streamer on the surface of the opposite electrode. From GB-2072051 it is known that preferably the minimum thickness is at least four times the spot radius of the streamer on the surface of the opposite electrode. The invention nevertheless is not limited to providing resistive material with such a minimum thickness.

Systems as described in the first and the second embodiment and in certain setups according to the third embodiment, as shown e.g. in FIG. 10, can be advantageously applied for treatment of solid materials, such as e.g. continuous or web-like materials. Such treatments may e.g. be plasma induced grafting, surface activation such as for example promoting adhesion, controlling surface energies, improving biocompatibility, enhancing performance of species present at the surface of the material, etc., surface cleaning and degreasing, plasma deposition such as e.g. coating with aerosols, sterilization, oxidation, reduction, cross-linking (carbonization), etching etc. Typical examples of properties of materials that can be changed using these methods are the hydrophilicity, hydrophobicity, surface energy, adhesion to other materials, colorability, surface electrical conductivity and biocompactibility. Systems as shown in FIG. 11 according to the third embodiment of the present invention may advantageously be used for gas cleaning, such as e.g. removal of contaminants in a gas flow or a mixed particle/gas flow like removal of low concentrations of volatile organic compounds in off-gases and odour control. The systems of the present invention also may be used in under water applications.

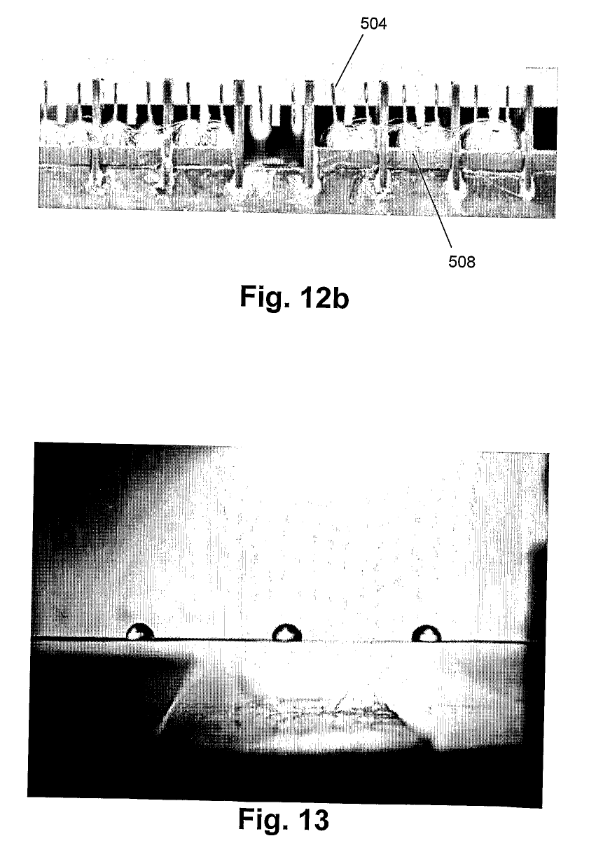

By way of illustration, the present invention not being limited thereto, advantages and effects obtainable with set-ups and methods according to certain embodiments of the present invention are illustrated using an experimental example of a surface treatment of a polypropylene strip. The experimental example is based on a system for treatment of material using a non-thermal plasma, as shown in FIG. 12a. FIG. 12b shows a photographic image of part of such a system. The system 500 comprises a plasma generating means comprising needle electrodes as first electrodes 504 and a resistive electrode as second electrode 508. The distance d between first electrodes 504 and second electrode 508 in the present example is substantially between 4.5 mm and 5 mm. In the present example, plasma generating means with different numbers of sections are used, i.e. a plasma generating means with a single section and a plasma generating means with a plurality of sections. The system of the present example also comprises a displacement means 510 based on air flow. It furthermore comprises a supporting means 514 adapted for supporting and moving the material 502 to be treated. In the present example, the material 502 to be treated is a polypropylene strip which is positioned at a distance s from the front side of the electrodes, i.e the side of the electrodes closest to the supporting means 514 as indicated in FIG. 12a. The treated polypropylene material 502 typically has a width w of substantially between 1.5 and 1.7 mm. Different parameters sets used in experiments according to the present example are shown by way of illustration in table 1.

TABLE 1

| | | Plasma generating |
|---|---|---|
| | Plasma generating | means with 6 |
| | means with 1 section | sections |
| Initial voltage | 16.4 kV-16.6 kV | 15.4 kV-15.7 kV |
| Working voltage | 15.9 kV-16.0 kV | 14.5 kV-14.6 kV |
| Air flow velocity | 22 m/s-50 m/s | 22 m/s-50 m/s |
| Average discharge current | 30 mA-35 mA | 180 mA-210 mA |
| Spark gap distance | 4.5 mm | 4.5 mm |
| Number of needle electrodes | 12 | 72 |
| Distance (d) resistive | 4.5 mm-5 mm | 4.5 mm-5 mm |
| electrode to needles | | |
| Distance (s) polypropylene to | 0.7 mm | 0.7 mm-1.9 mm |
| electrode plane (s) | | |

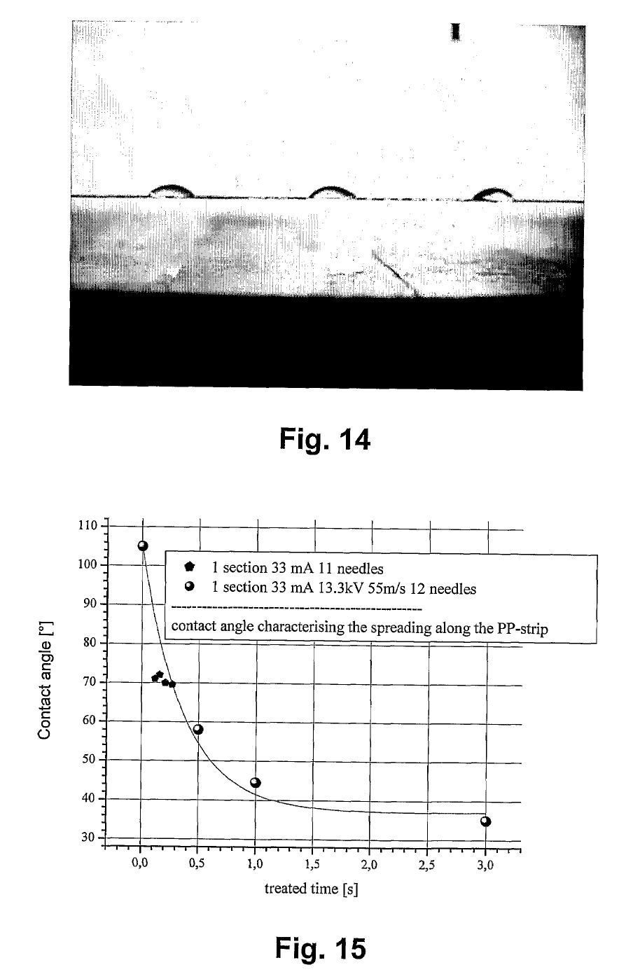

By way of illustration, experimental results are shown for the hydrophilicity of a polypropylene strip 502. The gas mixture used as processing gas has a substantial influence on both the composition of chemically active species generated by the non-thermal plasma (in the present case, by non-thermal plasma of streamers) and their life time. Varying the gas mixture allows changing the type of active species in order to obtain the optimal treatment effect for the specific material to be treated. For instance, by using ambient air as processing gas and varying its humidity, one can vary the number density of OH-radicals, which in some cases can play a crucial role in plasma processing of polymers and fabrics. The processing gas mixture can comprise O₂, N₂, H₂O, Ar, He, and different additives, changing the plasma-chemical reactions of active species with the surface to be treated. FIG. 13 and FIG. 14 respectively illustrate the difference in hydrophilicity for polypropylene that is not treated using non-thermal plasma in accordance with the present invention, and for polypropylene that has been subject to a non-thermal plasma treatment according to an embodiment of the present invention. FIG. 13 and FIG. 14 are photographs of three drops of liquid on the polypropylene surface. It can be seen that prior to treatment (FIG. 13), the polypropylene surface is less hydrophilic than after non-thermal plasma treatment according to an embodiment of the present invention. Measurements have shown that the contact angle for a liquid drop, e.g. a drop of distilled water, before treatment is about 104°, whereas the contact angle after treatment in accordance with an embodiment of the present invention is between 50° and 70°, whereby it is to be noticed that the smaller the contact angle is, the more hydrophilic the surface is.

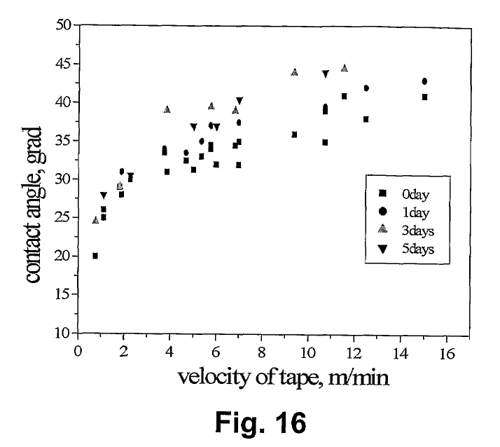

The effect of different treatment parameters on the contact angle, and thus the degree of treatment, is shown in FIG. 15 and FIG. 16.

FIG. 15 indicates the contact angle as a function of the treatment time for the material studied. The results are shown for a plasma generating means with one section of 11 needle electrodes and an average discharge current of 33 mA, indicated by five-sided polygonal symbols, and for a plasma generating means with one section of 12 needle electrodes, an average discharge current of 33 mA, a speed of 55 m/s and a working voltage of 13.3 kV, indicated with circular symbols. It can be seen that the longer the treatment time, the smaller the contact angle and thus the larger the degree of hydrophilicity.

FIG. 16 illustrates the contact angle as a function of the velocity of the material studied, i.e. of the polypropylene strip. It can be seen that the largest effects are obtained when the material to be treated has the smallest velocity, which may be expected as a smaller velocity results in a longer treatment time. The results are shown for a plasma generating means with one section of 12 needle electrodes, an average discharge current of 33 mA and a working voltage of 13.3 kV.

FIG. 16 also indicates the change of the contact angle as a function of the time after treatment and it can be seen that a substantially stable effect is obtained, although a small decrease in hydrophilicity could be noticed after a few days. In other words, the degradation of the plasma effect is only small.

It is to be understood that although preferred embodiments, specific constructions and configurations, as well as materials, have been discussed herein for devices according to the present invention, various changes or modifications in form and detail may be made without departing from the scope and spirit of this invention. For example, although the invention has been described with respect to the systems to be used for treatment of materials, the invention also relates to the corresponding methods of material treatment. The methods typically comprise creating a non-thermal plasma between a first and a second electrode and displacing the plasma from between the electrodes towards material to be treated. Displacing of the plasma is done such that a corresponding plasma current of the plasma interacting with the material is substantially parallel with the treatment area of the material to be treated. For continuous material, a rate of movement up to 10 m/s may be used, while still obtaining an efficient treatment of the material. Displacing may be done using any of the displacement means as described in the systems of the different embodiments or using any other suitable displacement means. Another method according to the present invention comprises selecting a type of discharge created in a plasma generating means by selecting a resistor value R and/or capacitor value C of an RC network coupled to at least one of the electrodes of the plasma generating means and then generating a plasma between the electrodes. A flow of material to be cleaned can be directed through the plasma, thus obtaining treatment of the material flow, or the plasma may be displaced from between the electrodes towards material to be treated, while the plasma still is pinned with edges to the electrodes of the system.

1-30. (canceled)

31. A device for non-thermal plasma treatment of a material at a pressure higher than 0.1 atmospheres, said device comprising:
a plasma generating means comprising at least a first electrode and a second electrode for generating a plasma current and a plasma pinned between said first electrode and said second electrode, and
a plasma displacement means for displacing part of said plasma current towards a longitudinally extending surface of an interaction area where said material is treated, so that said plasma current and said material interact and that a direction of said plasma current interacting with said material is substantially parallel with said surface of said interaction area where said material is treated.

32. A device according to claim 31, wherein said interaction area is a treatment area of said material to be treated.

33. A device according to claim 31, wherein at least one of said first electrode or said second electrode is positioned freely.

34. A device according to claim 31, wherein said plasma displacement means for displacing said plasma current towards an interaction area where said material is treated is an active means for displacing said plasma current towards an interaction area where said material is treated.

35. A device according to claim 32, wherein said device furthermore comprises a means for providing an area of said material to be treated in the vicinity of said electrodes.

36. A device according to claim 31, wherein said plasma displacement means comprises a flow material inlet system allowing to provide a flow material flowing substantially perpendicular to the direction of said plasma current.

37. A device according to claim 31, wherein said plasma displacement means comprises a means for generating an electric field.

38. A device according to claim 31, wherein said plasma displacement means comprises a means for generating a magnetic field.

39. A device according to claim 31, wherein said material to be treated is a solid, continuous web-like material.

40. A device according to claim 31, wherein said material to be treated has a relative rate of movement larger than 4 m/s with respect to said plasma generating means.

41. A device according to claim 31, wherein at least one of said first electrode or said second electrode is a resistive electrode.

42. A device according to claim 41, wherein said first electrode comprises a plurality of electrode elements.

43. A device according to claim 42, wherein said first electrode and said second electrode form a multi-pin to plate electrode system.

44. A device according to claim 42, wherein said plasma generating means furthermore comprises at least one RC network coupled to at least one of said electrode elements.

45. A device according to claim 44, wherein a resistor R in said RC network is selectable in order to select between different types of plasma discharge.

46. A device according to claim 45, wherein said resistor R is a variable resistor.

47. A device according to claim 31, wherein said interaction area is created by positioning a longitudinally extending surface of a plate in the vicinity of the electrodes, and wherein said material to be treated is a gaseous flow directed towards said plate.

48. A device according to claim 47, wherein said plate comprises a catalyst.

49. A device according to claim 31, wherein said plasma generating means comprises a DC high voltage generating source for generating said plasma in a DC power regime.

50. A device according to claim 31, wherein said plasma is a streamer-based plasma.

51. A method for non-thermal plasma treatment of a material at a pressure higher than 0.1 atmospheres, the method comprising:
creating a plasma discharge pinned between a first electrode and a second electrode of a plasma generating means, and
displacing said plasma discharge so that it interacts in an interaction area with said material to be treated and that a direction of said plasma current interacting with said material is substantially parallel with a surface of said interaction area.

52. A method according to claim 51, wherein said interaction area is a treatment area of said material to be treated.

53. A method according to claim 51, said method furthermore comprising providing said material to be treated near said electrodes.

54. A method according to claim 53, wherein providing said material to be treated near said electrodes comprises moving said material at a relative rate of movement with respect to said plasma generating means.

55. A method according to claim 51, wherein displacing said plasma discharge comprises generating any of an electric or a magnetic field near said plasma, said electric or magnetic field displacing part of said plasma discharge outside the region between said first electrode and said second electrode.

56. A method according to claim 51, wherein displacing said plasma discharge comprises providing a flow material flow onto said plasma substantially perpendicular to said plasma current.

57. A method according to claim 51, wherein said material to be treated is a gas, said interaction area is an area of a blocking material and a flow of said gas is directed to said blocking material.

58. A method according to claim 57, wherein said blocking material comprises a catalyst in said interaction area.

59. A method according to claim 57, wherein displacing is performed by said flow of said gas.

60. A method according to claim 51, wherein creating a plasma is performed in a DC power regime.

61. A device for high-pressure plasma treatment of a material, the device comprising a plasma generating means comprising at least a first electrode and a second electrode, at least one of them comprising a plurality of electrode elements, for generating a plasma between said electrode, wherein said plasma generating means comprises an RC network coupled to at least one electrode element of the first electrode or the second electrode.

62. A method for high-pressure plasma treatment of a material, the method comprising:
selecting at least one resistor value in at least one RC network coupled to at least one electrode element of a first electrode or a second electrode,
according to a type of plasma to be obtained, generating a plasma between at least a first electrode and a second electrode, at least one of said electrodes being connected to said RC network, and
providing said material to be treated thus allowing interaction between said material and said plasma.
